(19) United States
(12) Patent Application Publication
(10) Pub. No.: US 2012/0130222 A1
(43) Pub. Date: May 24, 2012

(54) MEASURING APPARATUS

(75) Inventors: Shuichi Kobayashi, Yokohama-shi (JP); Kazuhiko Fukutani, Yokohama-shi (JP)

(73) Assignee: CANON KABUSHIKI KAISHA, Tokyo (JP)

(21) Appl. No.: 13/292,255

(22) Filed: Nov. 9, 2011

(30) Foreign Application Priority Data
Nov. 19, 2010 (JP) 2010-258590

(51) Int. Cl. A61B 6/00

(52) U.S. Cl. 600/407

(57) ABSTRACT

A measuring apparatus is used, the apparatus including a probe having an element detecting an acoustic wave that has propagated through an object, an acoustic lens disposed between the probe and the object, and a signal processor obtaining object information from an electrical signal based on the acoustic wave detected by the element of the probe. The probe is disposed at a position where the element of the probe is made acoustically conjugate to a surface on a probe side of the object by the acoustic lens.

### BACKGROUND OF THE INVENTION

1. Field of the Invention

The present invention relates to a measuring apparatus.

2. Description of the Related Art

Research is being actively pursued in medical fields on measuring apparatuses that generate image data of spatial distributions of optical characteristics inside an object such as a living body using light irradiated from a light source such as laser. One of such measuring apparatuses is a PAT apparatus that utilizes photoacoustic tomography (PAT). In photoacoustic tomography, first, light is irradiated from a light source to an object to cause acoustic waves (typically, ultrasound) to be generated from a living tissue that has absorbed the energy of light that has propagated and diffused inside the object. The generated acoustic waves are received by a probe, that means acoustic wave detector, and the received signals are mathematically analyzed and processed to produce an image of spatial distribution information associated with optical characteristics of the inside of the object. This imaging procedure is referred to as image reconstruction.

A measuring apparatus for diagnosing breast cancer based on photoacoustic tomography using a pulsed laser light source that oscillates near-infrared light, which is a range of wavelengths having a high transmissivity to living bodies and thus called optical window, has been developed in recent years (see S. Manohar et al, Proc. of SPIE vol. 6437 643702-1).

The probe, or acoustic wave detector, needs to be in physical contact with the object in order to receive acoustic waves efficiently. Therefore the probe should preferably make direct contact with the object via liquid gel or the like that improves adhesiveness. However, if the object has a complex contour such as when the object is a small animal or a human breast, it is difficult to bring the receiving surface of the probe to complete contact with the surface of the object. In such a case, a shape maintaining member is used, such as a flat plate, for the purpose of flattening the shape of the object, for example, and the object is contacted with the probe via the shape maintaining member.

However, if such a shape maintaining member is used, the speed of sound inside the object and an average speed of sound through the shape maintaining member will be different. For this reason, the acoustic waves that have propagated through the object are refracted at an interface between the object and the shape maintaining member according to Snell's law. As a result, with normal image reconstruction based on photoacoustic tomography where the speed of sound is assumed to be constant, the reconstructed image has a reduced image resolution.

U.S. Patent Application Publication 2002/0173722 shows one method of solving the problem of effects of refraction at an interface. U.S. Patent Application Publication 2002/0173722 relates to a compound machine that combines X-ray mammography with an ultrasound diagnosis apparatus (apparatus that receives reflected ultrasound transmitted to and returned from inside an object). X-ray mammography generates image data by reconstructing an image from information on an object obtained by transmitting X-rays through the object compressed with a compression plate as a shape maintaining member. In the ultrasound apparatus combined with this X-ray mammography, the probe transmits and receives ultrasound via the compression plate.

Thus, time delays between a plurality of elements contained in the probe were calculated to perform a delaying process and the signals from respective elements were summed up so as to correct refraction of ultrasound waves caused by a difference in sound speed between the compression plate and the object.

The method of generating a three-dimensional image through image reconstruction in which refraction of ultrasound waves (acoustic waves) at the compression plate is corrected as disclosed in U.S. Patent Application Publication 2002/0173722 has the problem that it requires complex calculation and takes a long time for the arithmetic operations.

The probe is commonly an array probe having a plurality of sensor parts (elements) arranged one-dimensionally or two-dimensionally. The directionality of each element in an array probe is determined based on the shape and size of each element, and the characteristics of the range of acoustic waves. In image reconstruction based on photoacoustic tomography, information provided by acoustic waves from a wide range of directions will allow an image to be reconstructed with better reproducibility of the spatial information of optical characteristics inside the object.

However, with a commonly used planar array probe, the directionality of acoustic waves received by the probe is limited, leading to the problem that reconstructed images include artifacts therein.

### SUMMARY OF THE INVENTION

The present invention was devised in view of the problems described above, and an object of the invention is to provide a measuring apparatus capable of imaging with less degradation of resolution caused by refraction without performing complex arithmetic operations to correct the effects of refraction of acoustic waves.

This invention provides a measuring apparatus, comprising:

a probe including an element detecting an acoustic wave that has propagated through an object;

an acoustic lens disposed between the probe and the object;

a signal processor obtaining object information from an electrical signal based on the acoustic wave detected by the element of the probe,

wherein the probe is disposed at a position where the element of the probe is made acoustically conjugate to a surface on a probe side of the object by the acoustic lens.

According to the present invention, a measuring apparatus capable of imaging with less degradation of resolution caused by refraction without performing complex arithmetic operations to correct the effects of refraction of acoustic waves can be provided.

Further features of the present invention will become apparent from the following description of exemplary embodiments with reference to the attached drawings.

### BRIEF DESCRIPTION OF THE DRAWINGS

FIG. 1 is a schematic configuration diagram of the apparatus of Embodiment 1;

FIGS. 2A to 2C are diagrams for explaining signals received by a probe;

FIGS. 3A to 3C are diagrams for explaining signals used for image reconstruction;

FIG. 4A is a diagram for explaining refraction of acoustic waves at an object holding plate;

FIG. 4B is a diagram for explaining the function of an acoustic lens;

FIG. 5 is a schematic configuration diagram of the apparatus of Embodiment 2;

FIG. 6 is a summary diagram of essential parts of the apparatus of Embodiment 2;

FIGS. 7A and 7B are diagrams for explaining how directionality is overlaid in Embodiment 3;

FIG. 8 is a schematic configuration diagram of the apparatus of Embodiment 3; and

FIG. 9 is a schematic configuration diagram of the apparatus in another aspect of Embodiment 3.

### DESCRIPTION OF THE EMBODIMENTS

Hereinafter the measuring apparatus of the present invention will be described using the drawings.

The measuring apparatus of the present invention creates a conjugate image of probe elements on the surface on the probe side of an object. The apparatus performs signal processing based on the assumption that signals received by the probe elements were received at this position. The object information is then generated based on the results of the signal processing. The measuring apparatus of the present invention is typically a living body measuring apparatus designed for breasts, as will be described in the following description of embodiments. The measuring apparatus can also be termed as an object information acquiring apparatus that acquires object information from measurements.

In the following description, acoustic waves include those that are referred to as sound waves, ultrasound waves (elastic waves), and photoacoustic waves. Acoustic waves include the acoustic waves generated inside an object by irradiating light such as near-infrared light (electromagnetic waves) to the inside of the object, and reflected waves of acoustic waves transmitted into and returned from inside of an object.

The object measuring apparatus (object information acquiring apparatus) of the present invention includes an apparatus that uses an ultrasound echo technique wherein ultrasound is transmitted to an object and reflected waves (reflected ultrasound) reflected inside the object are received to obtain object information as image data or numerical data. The object information acquiring apparatus of the present invention also includes an apparatus that uses photoacoustic effects wherein acoustic waves (typically, ultrasound) generated inside an object by irradiating light (electromagnetic waves) to the object are received to obtain object information as image data or numerical data.

In the case with the former apparatus that uses an ultrasound echo technique, the object information that will be obtained is information reflecting differences in acoustic impedance of the tissues inside the object.

In the case with the latter apparatus that uses photoacoustic effects, the object information that will be obtained includes a distribution of sources of acoustic waves generated by irradiation of light, a distribution of initial pressures inside the object, or a distribution of absorbed optical energy densities deduced from the initial pressure distribution. The information also includes a distribution of absorption coefficients, a concentration distribution of a substance constituting a tissue, or absorption coefficients or concentrations of optical absorbers inside the object. The concentration distribution of substance may include, for example, an oxygen saturation distribution or a distribution of oxidized/reduced hemoglobin concentrations.

### Embodiment 1

Embodiment 1 of the present invention will be described below.

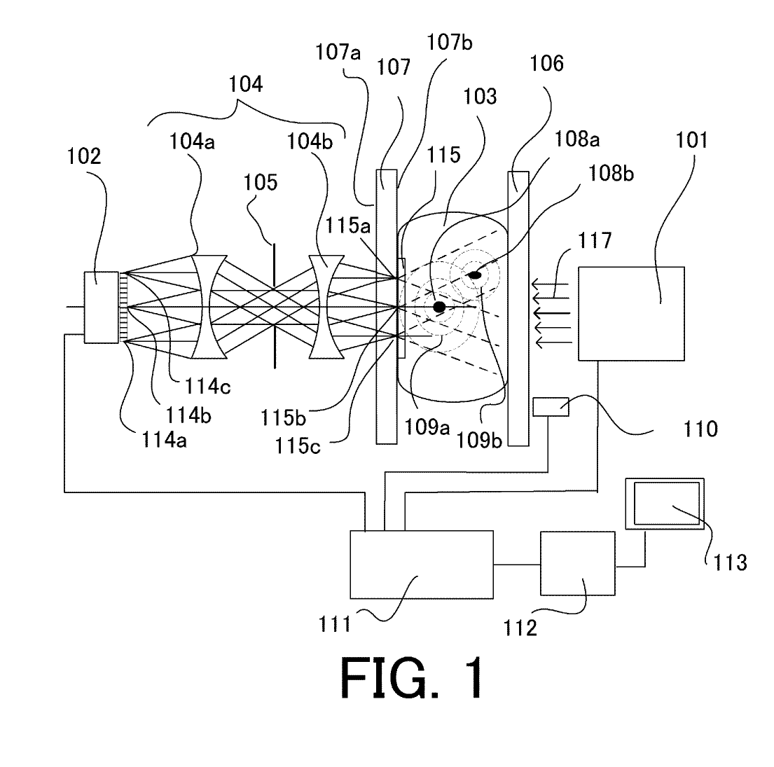

FIG. 1 is a schematic diagram showing the configuration according to Embodiment 1 of the present invention. The object measuring apparatus described in this embodiment is a living body measuring apparatus using photoacoustic tomography, which is a technique for reconstructing an image from received signals of acoustic waves generated inside an object by pulse irradiation of laser light.

The object in this embodiment is supposed to be a human breast. This apparatus performs imaging of blood vessels inside a breast using a photoacoustic tomography technique.

In FIG. 1, an illumination optical system 101 illuminates an object 103 with the light from a pulsed light source (not shown) that oscillates a wavelength in the near-infrared region with a predetermined optical energy density distribution. The object 103 is held between two object holding plates 106 and 107. The object holding plate 106 is located on the side of the illumination optical system 101 relative to the object. An interface between the surface of the object 103 and the object holding plate 107 will be referred to as 107b, and an interface on the opposite side will be referred to as 107a. A probe 102 is provided for receiving acoustic waves generated in the object 103. The object holding plate can also be termed as a holding unit holding an object.

The illumination light 117 irradiated from the illumination optical system 101 illuminates the object 103 through the object holding plate 106. The illumination light 117 diffuses and propagates through the object 103. Blood vessels 108a and 108b or the like in the object having a high light absorption coefficient (which will also be referred to as light absorber) absorb the illumination light 117 instantaneously and undergo thermal expansion, thereby generating acoustic waves 109a and 109b. Some of the generated acoustic waves 109 are received by the probe 102 via the object holding plate 107, acoustic lenses 104a and 104b, and an acoustic diaphragm 105.

A region of the object 103 near the interface with the object holding plate 107 has an acoustically conjugate relation with elements of the probe 102 via the acoustic lens 104. In other words, the acoustic lens 104 is disposed such that the elements of the probe 102 have an acoustically conjugate relation with the interface 107b between the object 103 and the object holding plate 107 (surface of the object 103 on the side of the probe 102). Matching is provided in the spaces between the probe 102 and the acoustic lens 104 and between the acoustic lens 104 and the object holding plate 107 so as to minimize acoustic loss. More specifically, matching members are disposed in these spaces.

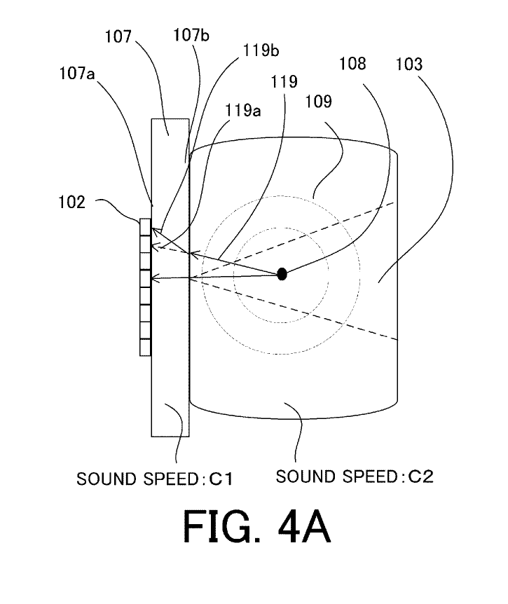

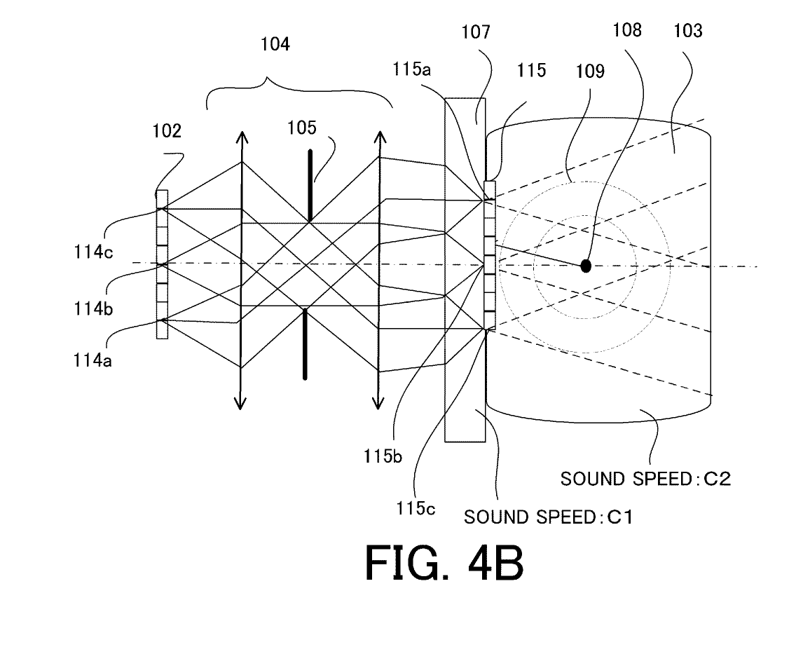

The function of the acoustic lens 104 will be described below. FIG. 4A shows how the acoustic waves 109 generated in a light absorber 108 such as a blood vessel in the object 103 propagate through the object holding plate 107.

The drawing shows a case where the probe 102 is disposed on the surface 107a of the object holding plate 107 on the opposite side from the object 103. This configuration has the following problem. Some of the acoustic waves 119 generated in the light absorber 108 proceeding toward the object holding plate refract at the interface 107b between the object 103 and the object holding plate 107. This is because the sound speed C1 in the object holding plate 107 is different from the sound speed C2 in the object 103. The refraction angle here is dependent on the incident angle of the acoustic wave incident to the object holding plate 107. Therefore, to work out the position of the light absorber, calculations in accordance with the incident angle are necessary, because of which the calculation load is very large.

FIG. 4B is a diagram schematically showing the arrangement of the probe 102 and the acoustic lens 104 in this embodiment. As shown in the drawing, a conjugate image of the probe 102 is formed by the acoustic lens 104 at an acoustically conjugate position 115 near the interface between the object holding plate 107 and the object 103 which is a living body. The acoustic lens 104 is designed in consideration of the effects of aberration caused by the refraction at the object holding plate 107.

With this configuration, positions 114a, 114b, and 114c on the probe 102 respectively form conjugate points at 115a, 115b, and 115c at the conjugate position 115 in the surface layer of the object.

The acoustic lens 104 should preferably be telecentric on the probe 102 side, taking into account the directionality of reception sensitivity of the probe 102.

Furthermore, in this embodiment, the lateral magnification at the conjugate position 115 was set at −1, and the lens was telecentric on the conjugate position 115 side as well.

Being telecentric means that the center line of converging beams of acoustic waves enters, for example, the probe 102 perpendicularly. That is, it means that the center line of the acoustic waves passed through the diaphragm surface and incident to the image surface is parallel to the axis of a center lens.

This configuration makes sensitivity characteristics parallel to each other at respective conjugate points 115a, 115b, and 115c of the conjugate position 115, so that it is as if the probe 102 were disposed at the conjugate position 115. Since the acoustic lens 104 corrects any aberration caused by refraction, correction of refraction shown in FIG. 4A is not necessary.

In FIG. 4B, refraction occurs at a plane interface with the object holding plate 107 on the side of the acoustic lens 104. This refraction at a plane surface can be corrected by changing the curvature of the acoustic lens 104. The acoustic lens 104 should preferably form an image of the probe 102 at the conjugate position 115 with little aberration and therefore the use of a non-spherical lens will be effective.

The method of producing images from signals received by the probe 102 will be described below.

As shown in FIG. 1, the illumination optical system 101 is electrically connected to a controller 111. A photodetector 110 is provided for detecting light emission timing of the pulsed light source (not shown) from the illumination optical system 101 and electrically connected to the controller 111. Similarly, the probe 102 is also electrically connected to the controller 111, and receives acoustic signals in synchronism with signals from the photodetector 110. A reconstructor (signal processor) 112 receives signals from the controller 111 and generates image data of a distribution of light absorbers such as blood vessels inside the object 103 which is a living body. A display unit 113 is a unit for displaying the image data.

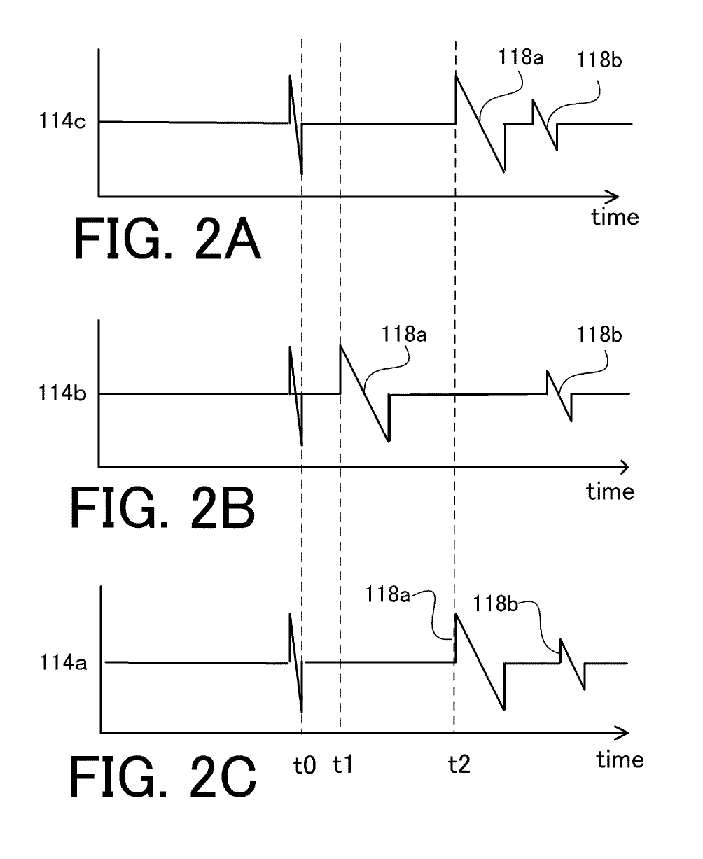

FIG. 2A shows signals received by the probe 102. This drawing shows an example of signals detected after the time when the photodetector 110 (FIG. 1) detected illumination light. In the drawing, t0 refers to the time when illumination light was detected. Photoacoustic signals emitted from the light absorbers 108a and 108b in FIG. 1 respectively correspond to the peaks 118a and 118b. FIG. 2A, FIG. 2B, and FIG. 2C respectively relate to signals received at detection points 114a, 114b, and 114c on the probe 102.

An acoustic wave generated at the interface reaches at time t0, due to the time required for the acoustic wave to propagate from a detection point on the probe to the interface 107b of the object holding plate 107 on the side of the object 103. There is a difference in peak detection time in accordance with the distance from respective elements to a light absorber. For example, the signal from the light absorber 108a is detected at the detection point 114b at time t1, while it is detected at the detection points 114a and 114c at time t2.

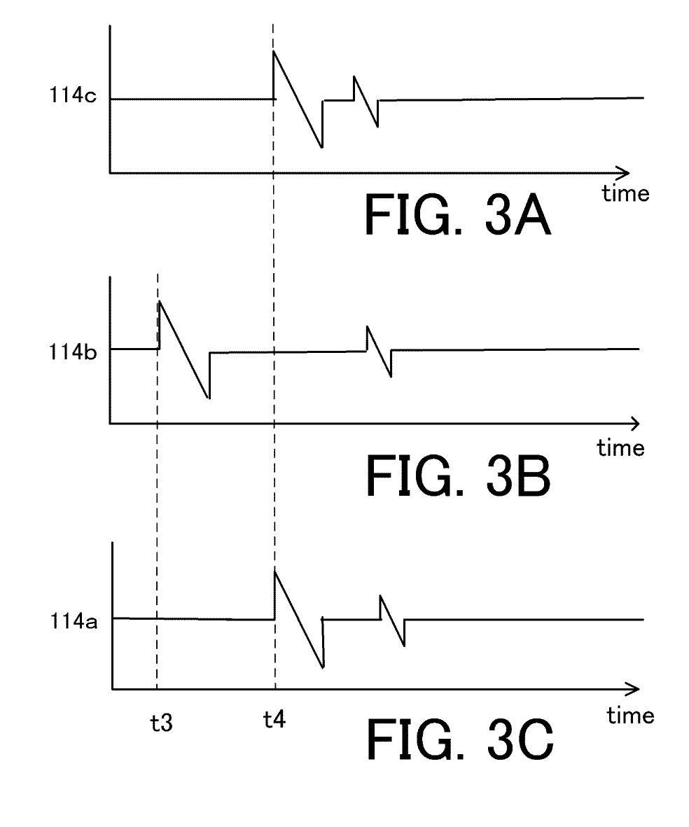

As shown in FIG. 3, data before t0 is deleted in the controller 111. Relations between t0, t1, and t2 in FIG. 2 and t3 and t4 in FIG. 3 are: t3=t1−t0 and t4=t2−t0. Signals thus obtained are utilized for image reconstruction. This removes any noise in the signals generated at the interface between the plate and the object.

It is also preferable to generate reconstructed image data by the reconstructor 112 after correcting intensity or the like of the signals of FIG. 3A, FIG. 3B, and FIG. 3C in consideration of acoustic propagation characteristics between the probe 102 and the conjugate position 115.

Acoustic matching should preferably be provided at the interfaces between the object holding plates 106 and 107 and the object 103. Preferable matching materials for this purpose include gel, urethane sheet, water and the like used in an ultrasound echo apparatus. For the object holding plate 107, materials having excellent ultrasound transmission characteristics such as polymethylpentene should preferably be used.

Furthermore, the material for the acoustic lens 104, and materials for filling the space between the acoustic lens 104 and the object holding plate 107 and the space between the acoustic lens 104 and the probe 102 should preferably be determined in consideration of acoustic matching. For example, for the acoustic lens 104, a resin material such as silicone rubber should preferably be used. The space should preferably be filled with oil or water. Taking into account the acoustic matching in this manner can reduce any acoustic wave loss caused by reflection at an interface.

The reconstructor 112 performs arithmetic operations to the electrical signals obtained by the controller 111 to generate reconstructed image data. A work station or the like is typically used as the reconstructor 112. The reconstructor performs a noise reduction process, correction associated with acoustic wave transmission between the conjugate position 115 and the probe 102, and the electrical signal offset correction mentioned above to the electrical signals received by the probe 102. Electrical signals thus corrected are then subjected to a reconstruction process.

Applicable reconstruction methods include time-domain and Fourier-domain back projection approaches commonly used in photoacoustic tomography techniques.

In this embodiment, the acoustic lens 104 was disposed as a telecentric system on the probe 102 side and on the conjugate position 115 side, with an acoustic image formation magnification of −1. This arrangement is equivalent to a system where the probe 102 is set at the conjugate position 115.

The acoustic image formation magnification may be set at a different value. This would mean that the respective sizes of elements on the probe 102 are changed by that magnification and therefore correction thereof would be necessary when reconstructing an image.

If the probe 102 is a planar array transducer, the lens should preferably be telecentric on the probe 102 side. In this case, the number of apertures (NA) would be designed in consideration of directionality of sensitivity of the probe 102 from an acoustic point of view, and so arranging the diaphragm 105 inside the acoustic lens 104 would be effective.

While the lens is telecentric on the conjugate position 115 side as well in this embodiment, the invention is not limited to this.

### Embodiment 2

Embodiment 2 of the present invention will be described below.

The object measuring apparatus described in this embodiment is basically a living body measuring apparatus using a photoacoustic tomography technique similarly to Embodiment 1. Here, however, the illumination optical system and the probe are configured movable so that the object can be scanned, whereby images of a wider field of view can be generated.

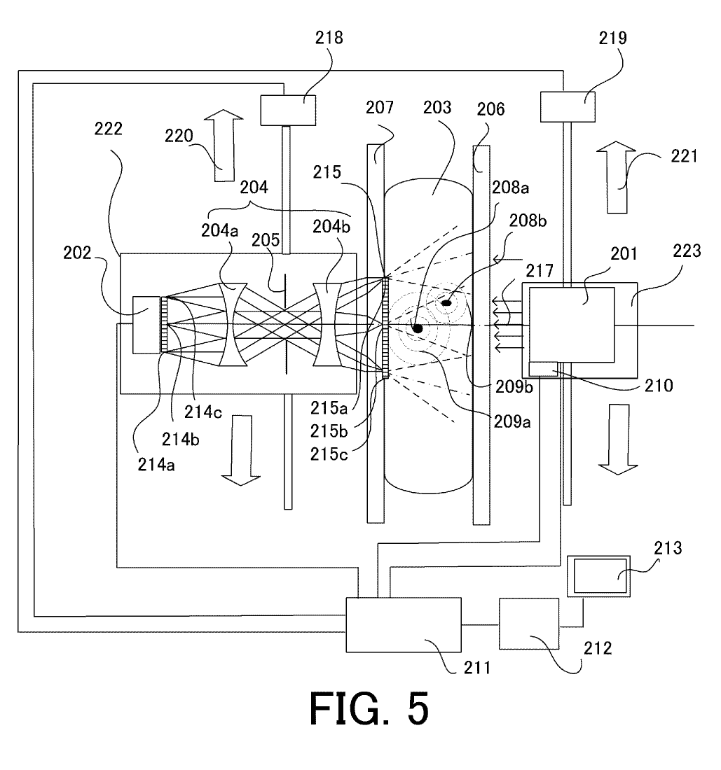

FIG. 5 is a schematic view showing the configuration according to Embodiment 2 of the present invention. The living body measuring apparatus of this embodiment obtains information of inside of an object using a photoacoustic tomography technique. Namely, the apparatus of this embodiment receives acoustic waves generated inside an object by pulse irradiation of laser light and reconstructs the received signals to obtain an image of a spatial distribution of absorption coefficients corresponding to the wavelength of the irradiated laser light.

The object in this embodiment is supposed to be a human breast. The wavelength of the laser light irradiated to the object is supposed to be near-infrared light. The living body measuring apparatus of this embodiment can perform imaging of blood vessels (blood) which are living tissues with a high absorption rate of the range of wavelengths of near-infrared light.

In FIG. 5, an illumination optical system 201 illuminates an object 203 with the light from a pulsed light source (not shown) that oscillates a wavelength in the near-infrared region with a predetermined optical energy density distribution. The object 203 is held between two object holding plates 206 and 207. The illumination light 217 from the illumination optical system 201 illuminates the object 203 through the object holding plate 206. The illumination light 217 diffuses and propagates through the object 203. Blood vessels 208a and 208b or the like in the object having a high light absorption coefficient absorb the illumination light 217 and undergo thermal expansion, thereby generating acoustic waves 209a and 209b. Some of the generated acoustic waves 209 are received by the probe 202 via the object holding plate 207, and further an acoustic diaphragm 205 and acoustic lenses 204a and 204b.

Similarly to Embodiment 1, because of the presence of the acoustic lens 204, the probe 202 has a conjugate relation with the interface 207b of the object holding plate 207 on the object side. Therefore, image reconstruction can be performed without taking into account the influence of aberration by refraction caused by a difference in sound speed between the object holding plate 207 and the object 203. The function of the acoustic lens 204 will not be described again as it is substantially the same as that of Embodiment 1.

Points 214a, 214b, and 214c on the probe 202 respectively form conjugate points 215a, 215b, and 215c on the surface of the object holding plate 207 on the object side via the acoustic lens 204. The magnification at the conjugate position 215 is set at −1.5 in this embodiment. This means that a probe that is geometrically 1.5 times larger is disposed at the conjugate position 215. Image reconstruction must be performed on the assumption of this fact.

Since the acoustic lens 204 should preferably be telecentric on the probe 202 side taking into account the directionality of reception sensitivity of the probe 202, the lens is set telecentric on the probe side in this embodiment.

The probe 202 and the acoustic lens 204 and others form a probe-side carriage 222 in this embodiment. The illumination optical system 201 and a photodetector 210 for detecting light irradiation from the illumination optical system 201 form an illumination optical system carriage 223.

The probe-side carriage 222 and the illumination optical system carriage 223 are mechanically connected to a carriage drive unit 218 and a carriage drive unit 219, respectively. The carriage drive units 218 and 219 are controlled by a controller 211. The carriage drive units 218 and 219 respectively move the probe 202 and the illumination optical system 201 to scan the positions on the object where photoacoustic signals are received, whereby a wider area than the probe 202 can be measured and imaged. Thus operation time can be reduced.

While the scanning directions of the probe-side carriage 222 and the illumination optical system carriage 223 are indicated by arrows 220 and 221 which run within the paper plane in this embodiment, the carriages may be configured to scan also in a direction perpendicular to the paper plane.

In this embodiment, a photoacoustic image over a wider area can be obtained by the scanning by the carriages 222 and 223 and synchronous control of the illumination by the illumination optical system 201 and timing of reception at the probe 202. Scanning by carriages may be performed continuously, or may be stopped and started repeatedly.

The method of producing images from signals received by the probe 202 will be described below.

The illumination optical system 201 is electrically connected to the controller 211. The photodetector 210 is provided for detecting light emission timing from the illumination optical system 201 and electrically connected to the controller 211. Similarly, the probe 202 is also electrically connected to the controller 211, and receives acoustic signals in synchronism with signals from the photodetector 210.

A reconstructor 212 receives signals from the controller 211 and generates image data of a distribution of light absorbers such as blood vessels inside the object 203 which is a living body. A display unit 213 is a unit for displaying the image data. Signals received by the probe 202 should preferably be corrected as required by offsetting the time required for propagation through the acoustic lens 204 as described in Embodiment 1 and in consideration of transmissivity or the like of the light path inside the acoustic lens 204.

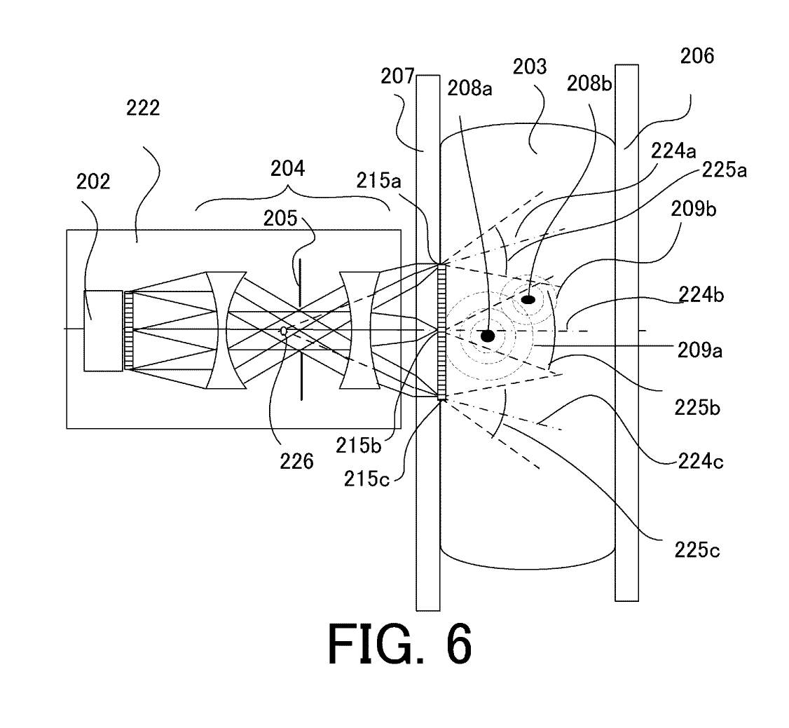

FIG. 6 shows the summary of essential parts of the apparatus according to this embodiment.

In this embodiment, the lens is not a telecentric system on the object holding plate 207 side. The exit pupil position 226 is set inside the acoustic lens 204 relative to the object holding plate 207, and chief rays 224a, 224b, and 224c are inclined.

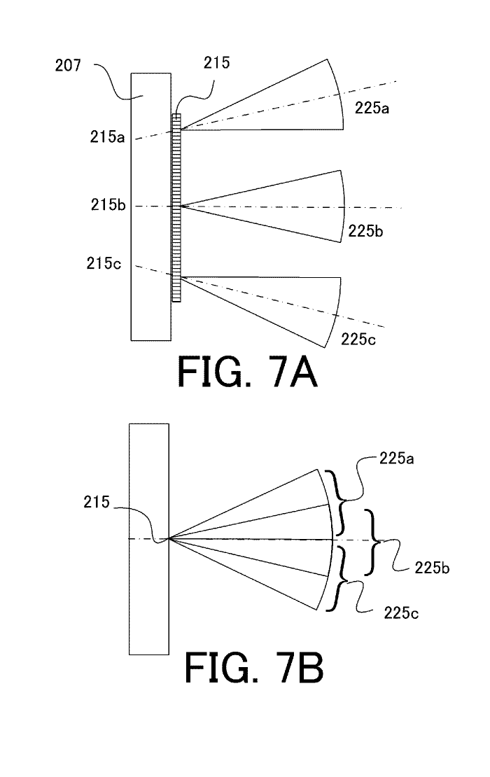

The apparatus of this embodiment, as described above, moves the probe-side carriage 222 for the scanning. FIG. 7A shows the directionality of probe sensitivity at the conjugate position 215 of the object holding plate 207. Assuming that elements of the probe 202 themselves have an angle of α° as a directionality angle, the conjugate image will have a directionality angle of α/1.5°, if the magnification at the conjugate position is set at 1.5.

While the angles of directionality at the conjugate points are reduced as compared to the elements of the probe, the directionality angles 225a, 225b, and 225c at the conjugate points are oriented to different directions. This provides an effect of increasing the directionality angle as the angles are overlaid as shown in FIG. 7B.

Receivable signal directions can thus be increased by the setting of the exit pupil position 226, which enables reception of acoustic waves from a wider angle, so that noise in the reconstructed image can be reduced.

Similarly to Embodiment 1, acoustic matching should preferably be provided at the interfaces between the object holding plates 206 and 207 and the object 203. Preferable matching materials for this purpose are the same as those of Embodiment 1. Preferable materials for the object holding plate 207 are the same as those of Embodiment 1, too.

The material for the acoustic lens 204, and materials for filling the space between the acoustic lens 204 and the object holding plate 207 and the space between the acoustic lens 204 and the probe 202 should preferably be determined in consideration of acoustic matching. Preferable materials for these components are the same as those of Embodiment 1. Taking into account the acoustic matching in this manner can reduce any acoustic wave loss caused by reflection at an interface.

The reconstructor 212 performs arithmetic operations to the electrical signals obtained by the controller 211 similarly to Embodiment 1 to generate reconstructed image data (not shown). The processes the reconstructor performs and reconstruction methods are the same as those of Embodiment 1.

If the probe 202 is a planar array transducer, the lens should preferably be telecentric on the probe 202 side. In this case, the number of apertures (NA) would be designed in consideration of directionality of sensitivity of the probe 202 from an acoustic point of view, and so arranging the diaphragm 205 inside the acoustic lens 204 would be effective.

While the acoustic image formation magnification at the conjugate point 215 relative to the probe 202 was described as −1.5 in this embodiment, the magnification may be set so as to reduce the probe, e.g., at 0.5. Reducing the image formation magnification at the conjugate point 215 relative to the probe 202 is expected to provide similar effects as reducing the size of the aperture of the probe and can improve the resolution power. Not to mention, reconstruction must be performed on the assumption of this fact. The magnification may be set at a different value.

### Embodiment 3

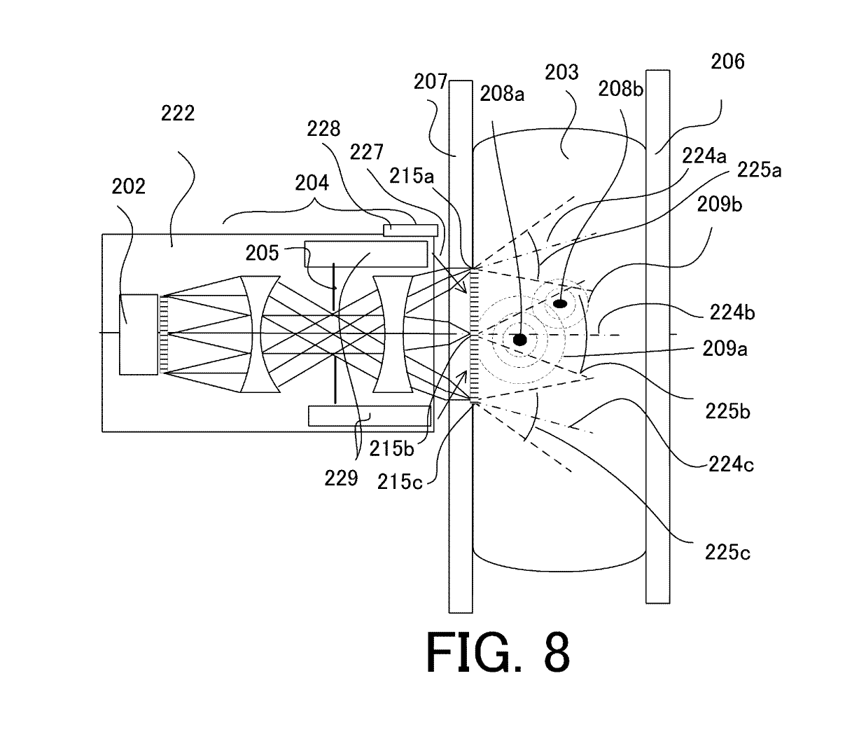

Embodiment 3 of the present invention will be described below. FIG. 8 is a diagram showing the summary of essential parts of the apparatus according to Embodiment 3. Same numbers are given to the components having the same functions as those of the apparatus of FIG. 5 and will not be described again. The apparatus of this embodiment has a configuration in which a probe-side illumination optical system 229 and a photodetector 228 are disposed inside the probe-side carriage 222 of the apparatus according to Embodiment 2. Similarly to Embodiment 2, the apparatus of this embodiment is an apparatus for obtaining reconstructed images based on the principles of photoacoustic tomography.

The apparatus of this embodiment illuminates an object 203 with illumination light 227 from the illumination optical system 229 and the photodetector 228 captures timing of the light illumination.

Arranging the illumination optical system 229 inside the probe-side carriage 222 enables the object 203 to favorably receive signals from the probe 202 side. While the illumination optical system carriage in FIG. 5 is omitted in this embodiment, the carriage may be used in combination, which will enable formation of images of deep inside the living body.

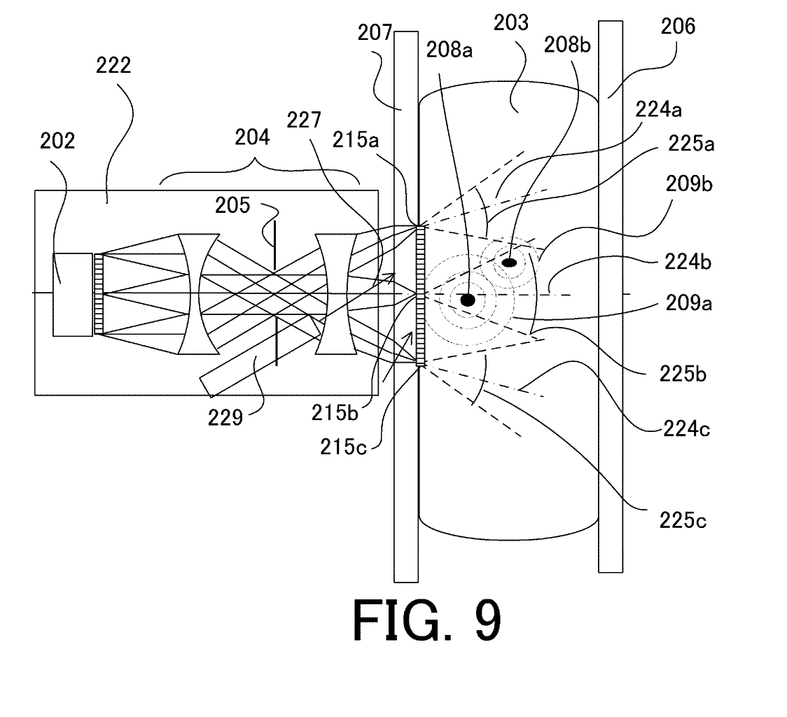

FIG. 9 shows another aspect of this embodiment. Same numbers are given to the components having the same functions as those of the apparatus of FIG. 8 and will not be described again.

The apparatus of this drawing has a configuration in which illumination light 227 from the illumination optical system 229 disposed on the probe side transmits part of the acoustic lens 204. This arrangement enables efficient illumination of a reception region of the probe 202.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-258590, filed on Nov. 19, 2010, which is hereby incorporated by reference herein in its entirety.

What is claimed is:

1. A measuring apparatus, comprising:
a probe including an element detecting acoustic wave that has propagated through an object;
an acoustic lens disposed between the probe and the object;
a signal processor obtaining object information from an electrical signal based on the acoustic wave detected by the element of the probe,
wherein the probe is disposed at a position where the element of the probe is made acoustically conjugate to a surface on a probe side of the object by the acoustic lens.

2. The measuring apparatus according to claim 1, further comprising a holding unit disposed between the object and the acoustic lens and holding the object,
wherein the probe is disposed at a position where the element of the probe is made acoustically conjugate to a surface of the object at an interface with the holding unit by the acoustic lens.

3. The measuring apparatus according to claim 1, wherein the acoustic lens is disposed so as to be telecentric on a side of the probe.

4. The measuring apparatus according to claim 2, further comprising a driving unit for moving the probe on the holding unit,
wherein the probe detects an acoustic wave at respective positions to which the probe has been moved by the unit.

5. The measuring apparatus according to claim 1, wherein the acoustic wave that has propagated through the object is a photoacoustic wave generated when the object is irradiated with light.

6. The measuring apparatus according to claim 1, wherein the acoustic wave that has propagated through the object is an elastic wave transmitted to the object and reflected inside the object.
